(19) United States
(12) Patent Application Publication
(10) Pub. No.: US 2018/0098756 A1
(43) Pub. Date: Apr. 12, 2018

(54) BIOPSY SYSTEM AND METHOD OF USE

(71) Applicant: COVIDIEN LP, Mansfield, MA (US)

(72) Inventors: JUSTIN GRUMBIR, MINNETONKA, MN (US); MICHAEL J. KERN, MINNETONKA, MN (US)

(21) Appl. No.: 15/291,699

(22) Filed: Oct. 12, 2016

(51) Int. Cl. A61B 10/02

(52) U.S. Cl. CPC A61B 10/0266

(57) ABSTRACT

A needle biopsy system includes an elongated shaft and a needle shaft. The elongated shaft includes a proximal portion, an intermediate portion more flexible than the proximal portion, and a distal portion that is less flexible than the intermediate portion. The needle shaft has a distal tip configured to cut and receive a sample of tissue and is longitudinally movable through the elongated shaft between a first position and a second position. In the first position, the distal tip of the needle shaft is disposed within the elongated shaft, and in the second position, the distal tip of the needle shaft extends distally from the distal portion of the elongated shaft to cut and receive a sample of tissue.

### BACKGROUND

### 1. Technical Field

The present disclosure relates to a surgical system for taking tissue samples and, more specifically, to surgical systems for performing needle aspiration biopsies of tissue.

### 2. Discussion of Related Art

Needle biopsy is a medical procedure used to obtain a tissue sample from an area of the body. The tissue sample is usually tested to assist in diagnosing a medical condition or to assess the effectiveness of a particular treatment. Percutaneous needle lung biopsy or transthoracic needle lung biopsy involves the use of a needle to enter the lung through the skin to obtain a biopsy sample. During lung biopsies, great care is taken to avoid inadvertent puncturing of the lung, which may lead to bleeding and/or lung collapse due to leakage from the lung. Typically, bleeding and lung collapse are more likely with larger, relatively stiff needles and/or flat-tipped needles. However, using a needle having a relatively small diameter may be undesirable because the sample obtained using such a small needle may be insufficient for histological examination.

Typically, biopsy needles are only one component of a biopsy system. For example, a biopsy system used for percutaneous lung biopsies may include a biopsy needle having a flexible shaft and a flexible access catheter through which the biopsy needle gains entry into a target tissue site in the lung. Such a biopsy system is easily navigable through the various narrow passageways of the lung due to the flexibility of the access catheter and the flexibility of the biopsy needle, which facilitates conformance of these components to the deviating passageways of the lung.

Accordingly, the components of a system used for percutaneous lung biopsy should be designed to minimize the chance of lung collapse, have sufficient flexibility for navigation through the deviating passageways of the lung, improve the sample size of a biopsy, and also maximize the ability to pierce the skin and other tissue while minimizing the chance of bleeding.

### SUMMARY

This disclosure relates generally to a needle biopsy system that includes an elongated shaft and a needle shaft. The elongated shaft includes a proximal portion, an intermediate portion more flexible than the proximal portion, and a distal portion that is less flexible than the intermediate portion. The needle shaft has a distal tip configured to cut and receive a sample of tissue and is longitudinally movable through the elongated shaft between a first position and a second position. In the first position, the distal tip of the needle shaft is disposed within the elongated shaft, and in the second position, the distal tip of the needle shaft extends distally from the distal portion of the elongated shaft to cut and receive a sample of tissue.

In some embodiments, the elongated shaft may have an outer tubular surface, an inner tubular surface, and a braiding extending longitudinally between the outer tubular surface and the inner tubular surface.

It is contemplated that the braiding may extend along the proximal and distal portions of the elongated shaft and terminate proximally of the distal portion of the elongated shaft. The braiding may include a plurality of interwoven metallic braid filaments.

It is envisioned that the inner tubular surface may have a constant durometer along its length. The outer tubular surface may have a higher durometer along the proximal portion of the elongated shaft than at the intermediate portion and the distal portion of the elongated shaft.

In some embodiments, the proximal portion of the elongated shaft may be less flexible than the intermediate portion and the distal portion of the elongated shaft.

It is contemplated that the distal portion of the elongated shaft may be tapered in a distal direction.

It is envisioned that the biopsy system may further include a first hub coupled to the proximal portion of the elongated shaft and a second hub coupled to a proximal portion of the needle shaft. The first hub may be configured to receive the needle shaft therethrough. The needle shaft may be configured to move through and relative to the first hub in response to movement of the second hub. The first hub may have a proximal portion configured to detachably engage a distal portion of the second hub, and the second hub may be configured for a fluid tight connection with a syringe.

In some embodiments, the biopsy system may further include a stylet configured to move longitudinally within a lumen defined through the needle shaft. The stylet may include a blunt distal tip configured to be disposed within the distal tip of the needle shaft. The stylet may be less flexible than the intermediate portion of the elongated shaft.

Also provided by the present disclosure is a method of performing a needle biopsy of lung tissue. The method includes flexing a portion of an elongated shaft to facilitate moving the elongated shaft through an airway of a lung and positioning a blunt distal portion of the elongated shaft adjacent tissue in the lung. An intermediate portion of the elongated shaft is more flexible than a proximal portion of the elongated shaft, and the blunt distal portion is less flexible than the intermediate portion and more flexible than the proximal portion. The method further includes moving a needle shaft longitudinally within the elongated shaft to extend a distal tip of the needle shaft from the distal portion of the elongated shaft to cut and receiving a sample of tissue.

In some embodiments, the method may further include moving a blunt distal tip of a stylet in a proximal direction to a position proximal of the distal tip of the needle shaft.

It is contemplated that the method may include moving a stylet longitudinally within a lumen defined through the needle shaft to position a distal tip of the stylet within the distal portion of the needle shaft.

Further, to the extent consistent, any of the aspects described herein may be used in conjunction with any or all of the other aspects described herein.

### BRIEF DESCRIPTION OF THE DRAWINGS

Various aspects of the present disclosure are described hereinbelow with reference to the drawings, which are incorporated in and constitute a part of this specification, wherein:

FIG. 1 is a schematic illustration of a biopsy system including a catheter assembly and a needle assembly used for percutaneous removal of a sample of tissue;

FIG. 2 is a cross-sectional view, taken along line 2-2 of FIG. 1, illustrating a needle shaft disposed within an elongated shaft of the catheter assembly;

FIG. 3 is a side view of the elongated shaft of FIG. 1;

FIG. 4 is an enlarged view of detail 4 of FIG. 3 illustrating a distal portion of the elongated shaft;

FIG. 5 is a cross-sectional view, taken along line 5-5 of FIG. 4, of the distal portion of the elongated shaft;

FIG. 6 is a cross-sectional view, taken along line 6-6 of FIG. 3, illustrating a plurality of layers of the elongated shaft;

FIG. 7 is a side view of braiding of the elongated shaft of FIG. 6;

FIG. 8 is a schematic illustration of the biopsy system of FIG. 1 illustrating a distal tip of the needle shaft extending distally from the elongated shaft;

FIG. 9 is an enlarged view of detail 9 of FIG. 8;

FIG. 10A is an enlarged side view of the distal tip of the needle shaft of FIG. 9;

FIG. 10B is a cross-sectional view, taken alone line 10B-10B of FIG. 10A, of the distal tip of the needle shaft of FIG. 9;

FIG. 10C is an enlarged top view of the distal tip of the needle shaft of FIG. 9;

FIG. 10D is an enlarged view of detail 10D of FIG. 10B illustrating threading of a proximal portion of the distal tip of the needle shaft of FIG. 9;

FIG. 11A is a partial side view of the biopsy system of FIG. 1 including a stylet disposed in a first position;

FIG. 11B is a side view of the biopsy system of FIG. 11A illustrating the stylet disposed in a second position;

FIG. 12A is a side view of the stylet of FIG. 11A;

FIG. 12B is a front view of the stylet of FIG. 11A; and

FIG. 12C is an enlarged view of detail 12C of FIG. 12A illustrating a hub of the stylet.

### DETAILED DESCRIPTION

Embodiments of the disclosed biopsy system and method of use are described with reference to the accompanying drawings. Like reference numerals may refer to similar or identical elements throughout the description of the figures. As shown in the drawings and as used in this description, the term “proximal” refers to that portion of the described biopsy system including the catheter assembly, the needle assembly, or the stylet, that is closer to the user, and the term “distal” refers to that portion of the biopsy system including the catheter assembly, the needle assembly, or the stylet, that is farther from the user.

Reference will now be made in detail to embodiments of the present disclosure. While certain exemplary embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the scope of the embodiments of the present disclosure as defined by the appended claims.

The present disclosure provides a biopsy system for percutaneous removal of a sample of tissue, for example, tissue in the lung. The biopsy system generally includes a catheter assembly and a needle assembly. The needle assembly includes a needle shaft movably disposed within an elongated shaft of the catheter assembly. In some embodiments, a stylet is movably disposed within the needle shaft. The needle shaft has a proximal portion, a distal portion, and a distal tip that is configured for cutting and receiving a sample of tissue. The biopsy system has sufficient flexibility to facilitate navigation through passageways of the lung. More specifically, the flexibility of any one of the proximal portion, the distal portion, or the distal tip of the needle shaft may differ or be the same relative to each other. For example, the distal portion may be more flexible than the proximal portion, less flexible than the proximal portion, or have the same flexibility as the proximal portion. In either of the above described examples, the distal tip may be less flexible than either of the distal or proximal portions, more flexible than either of the distal or proximal portions, or have the same flexibility as either of the distal or proximal portions.

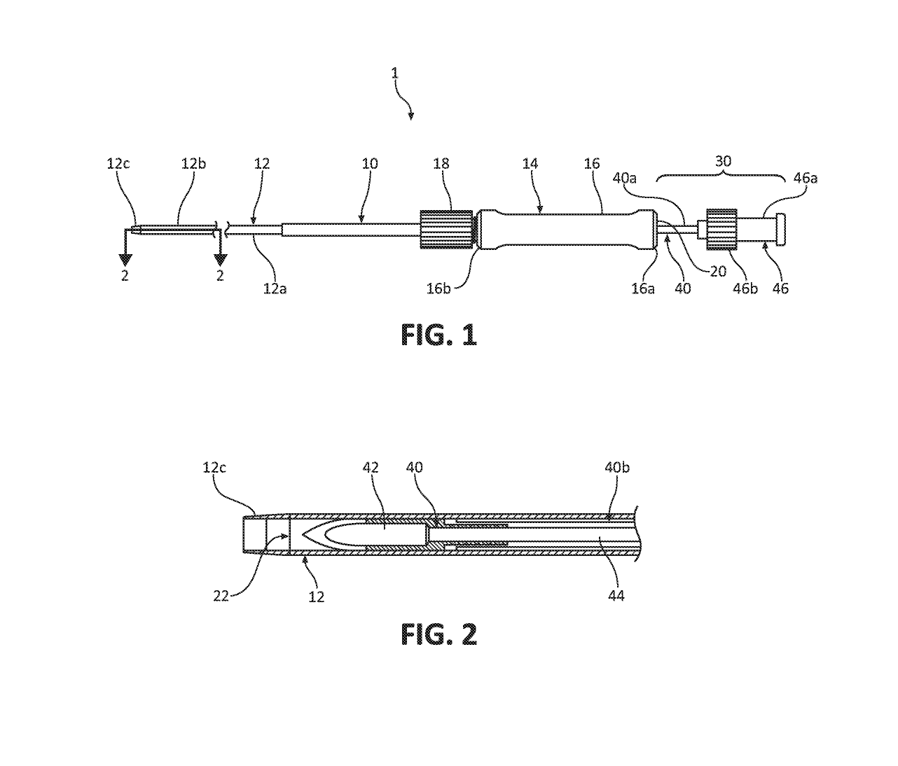

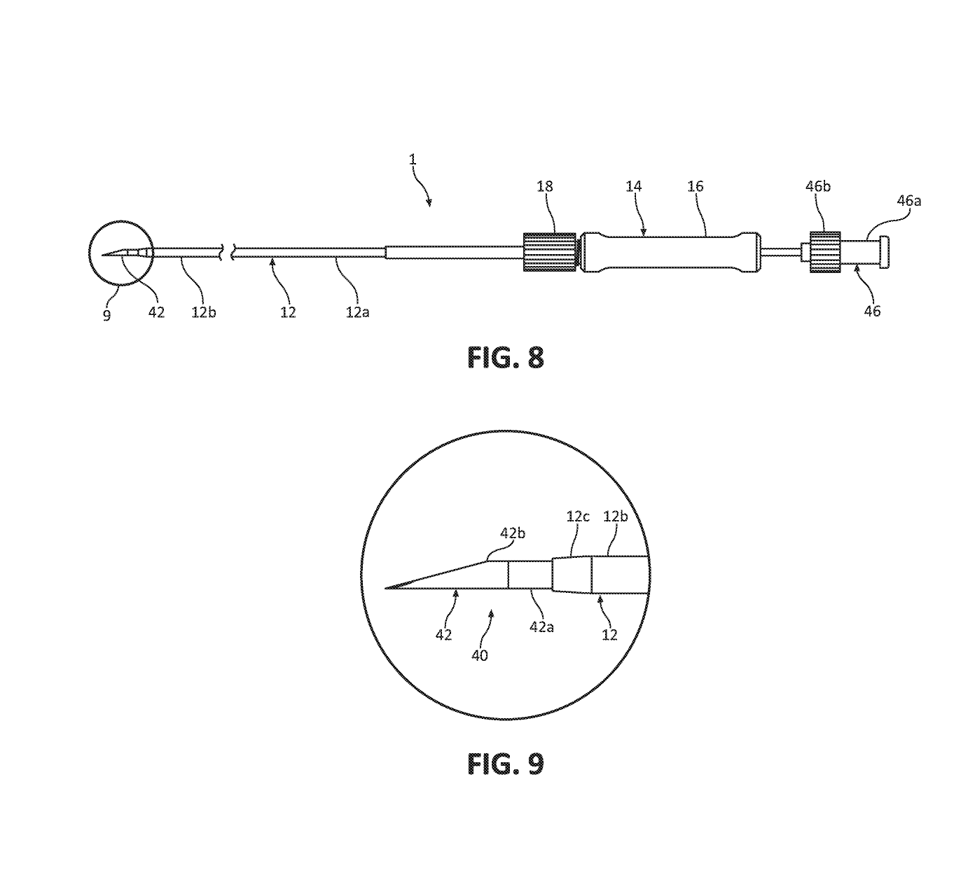

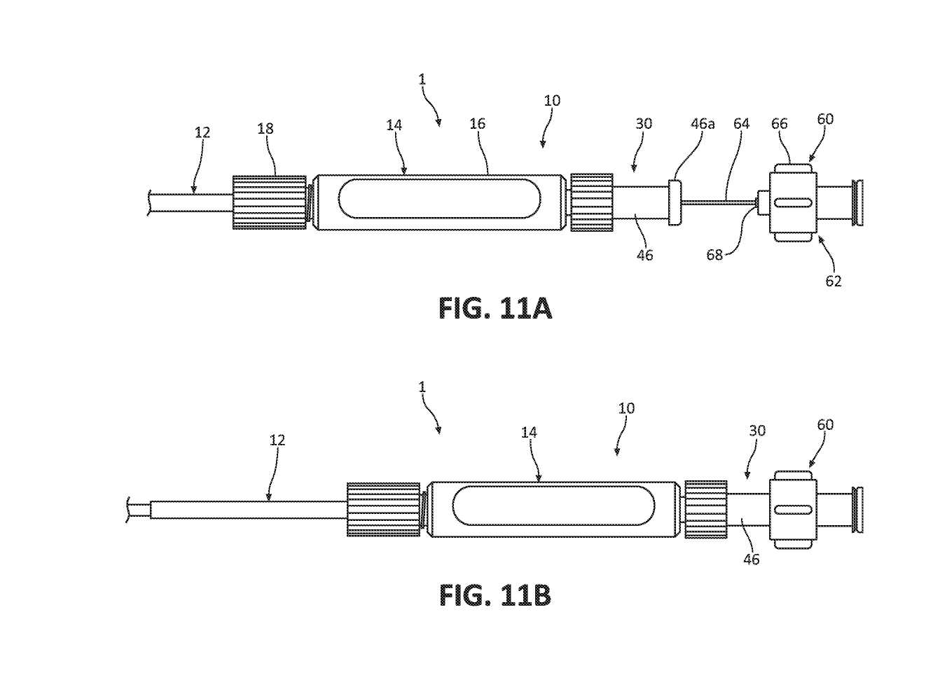

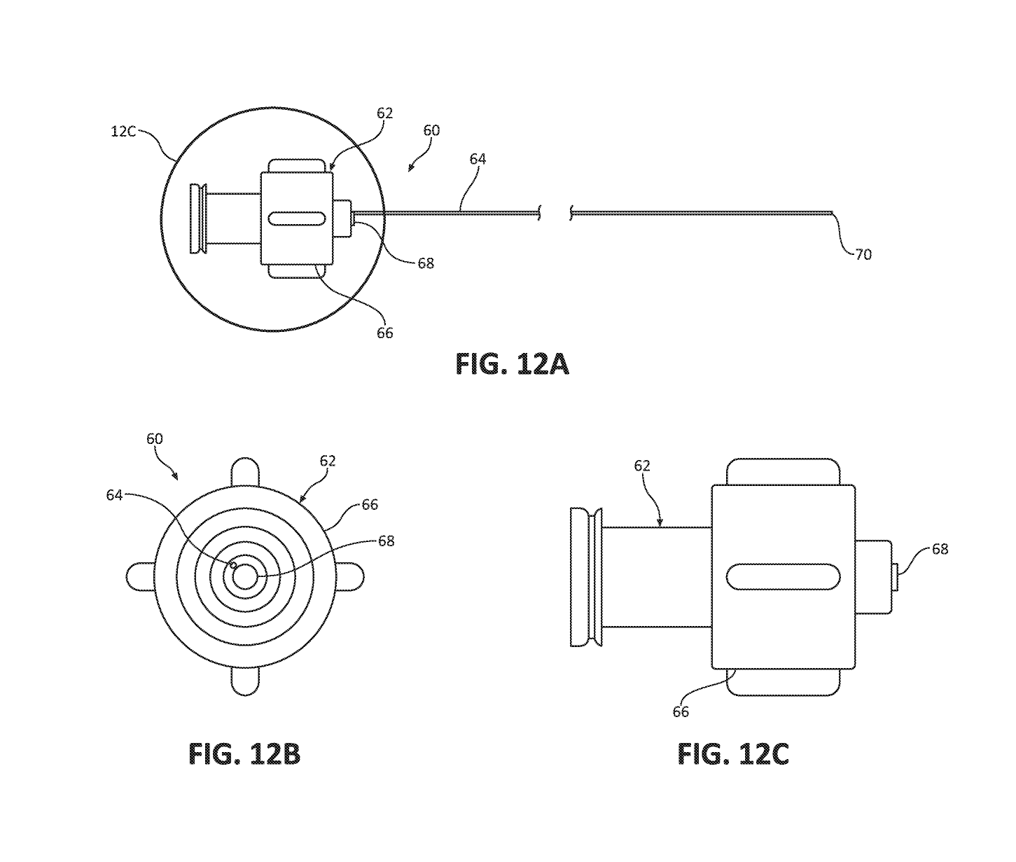

With reference to FIGS. 1, 2, 8, and 9, a biopsy system 1 is provided for percutaneous removal of a tissue sample, for example, lung tissue. Biopsy system 1 generally includes a catheter assembly 10, a needle assembly 30, and in some embodiments (see FIGS. 11A-12C) a stylet 60. The catheter assembly 10 includes an elongated shaft 12 extending distally from a hub 14. The elongated shaft 12 has a proximal portion 12a, an intermediate portion 12b, and a distal portion 12c. The needle assembly 30 includes a needle shaft 40 having a distal tip 42 and extending distally from a hub 46. The elongated shaft 12 of the catheter assembly 10 may be, for example, a catheter, a cannula, a tube, or the like. The needle shaft 40 of the needle assembly 30 is longitudinally movable through the elongated shaft 12 of the catheter assembly 10 to move the distal tip 42 of the needle shaft 40 from a first position disposed within the elongated shaft 12, as shown in FIGS. 1 and 2, to a second position deployed from the distal portion 12c of the elongated shaft 12 for cutting and receiving tissue, as shown in FIGS. 8 and 9.

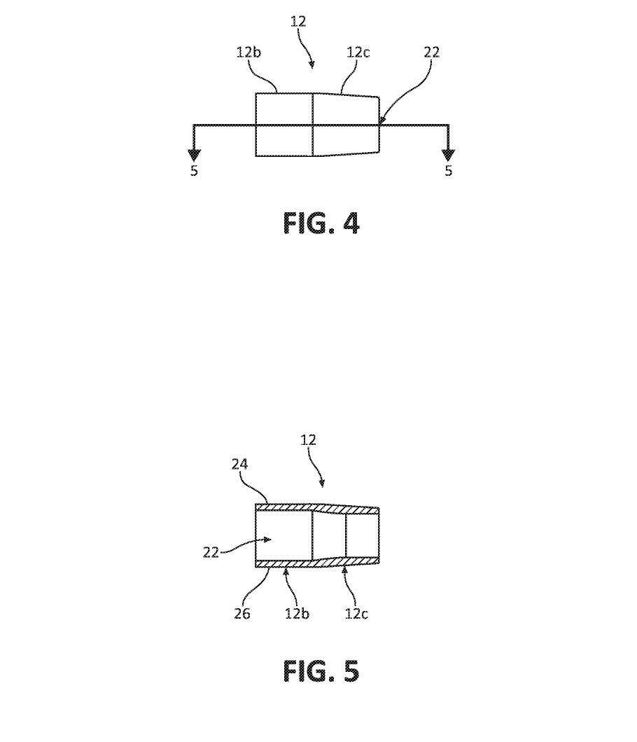

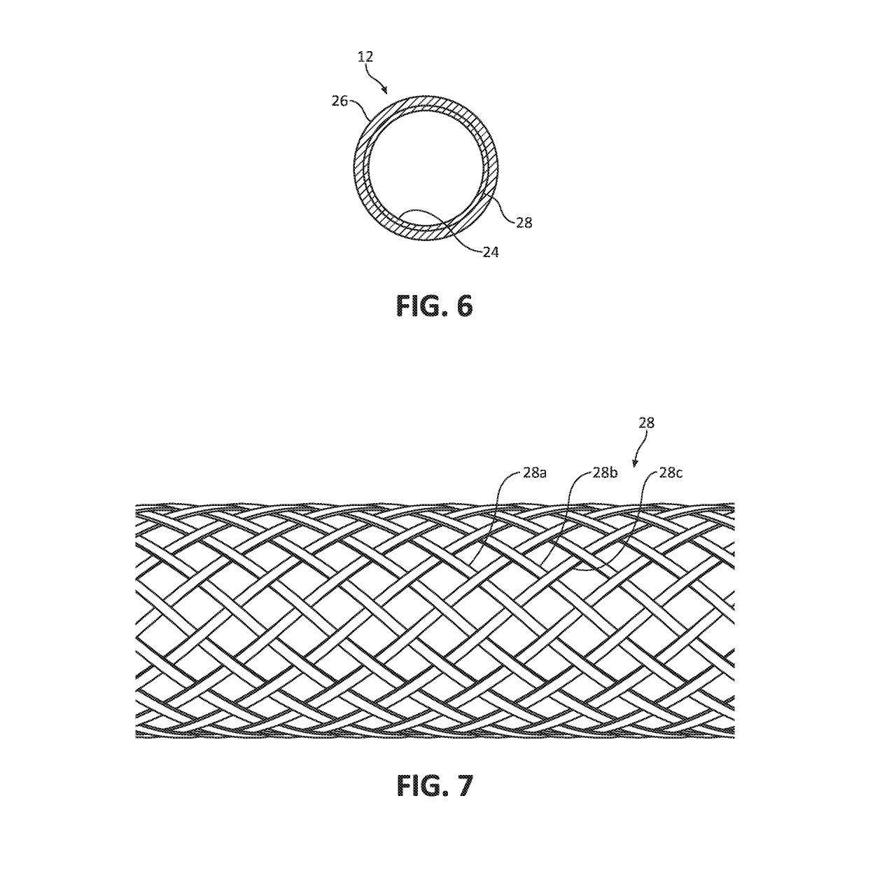

With reference to FIGS. 1-7, hub 14 of catheter assembly 10 includes an elongated handle portion 16 and a connector cap 18. Handle portion 16 of hub 14 is configured to be grasped by a hand of a clinician during use of biopsy system 1. Handle portion 16 has a distal portion 16b and a proximal portion 16a configured to be detachably coupled to hub 46 of needle assembly 30. Connector cap 18 of hub 14 is attached to distal portion 16b of handle portion 16 and is configured to couple handle portion 16 to proximal portion 12a of elongated shaft 12.

Although not explicitly shown, in some embodiments, connector cap 18 includes threading (e.g., female threading) defined therein for threading engagement to corresponding threading (e.g., male threading) defined on proximal portion 12a of elongated shaft 12. Additionally, it is contemplated that hub 14 may be coupled to proximal portion 12a of elongated shaft 12, either permanently or detachably, via any suitable fastening engagement, for example, compression fit, friction-fit, interference fit, snap-fit, adhesives, or the like.

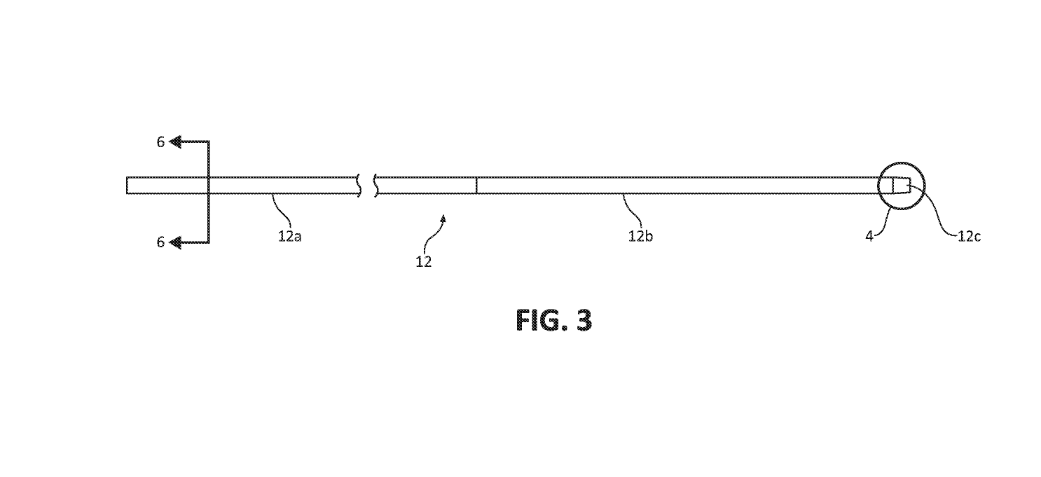

With reference to FIGS. 3-7, proximal portion 12a, intermediate portion 12b, and distal portion 12c of elongated shaft 12 of catheter assembly 10 may differ in flexibility relative to each other. For example, in some embodiments, proximal portion 12a of elongated shaft 12 has a first flexibility, intermediate portion 12b of elongated shaft 12 has a second flexibility that is greater than the first flexibility of proximal portion 12a, and distal portion 12c has a third flexibility that is less than the second flexibility of intermediate portion 12b. The first flexibility of proximal portion 12a may be less than each of the second and third flexibilities of distal portion and distal tip 12b, 12c, respectively.

Elongated shaft 12 of catheter assembly 10 may have a total overall length of about 1300 mm to about 1500 mm and, in some embodiments, about 1400 mm. Intermediate portion 12b may have an overall length of about 40 mm to about 60 mm and, in some embodiments, about 52 mm. Distal portion 12c may have an overall length of about 1 mm to about 3 mm and, in some embodiments, about 2 mm. Distal portion 12c tapers in a distal direction and terminates in a blunt, distal face configured for atraumatic insertion through tissue. The tapering of distal portion 12c provides for a smooth transition from distal portion 12c of elongated shaft 12 to distal tip 42 of needle shaft 40 when distal tip 42 is deployed from distal portion 12c of elongated shaft 12, as shown in FIGS. 8 and 9. In some embodiments, distal portion 12c may terminate in a sharpened tip configured to pierce tissue.

With continued reference to FIGS. 3-7, elongated shaft 12 of catheter assembly 10 includes an inner tubular surface 24 and an outer tubular surface 26. Inner tubular surface 24 is disposed within the outer tubular surface 26 and defines a longitudinally-extending inner lumen 22 (FIGS. 2, 5, and 6) therein configured to slidably receive needle shaft 40 therethrough. Inner lumen 22 is in fluid communication with a longitudinal passageway 20 defined through handle portion 16 of catheter assembly 10. In some embodiments, inner tubular surface 24 may have a substantially constant durometer along its length and is in the form of a plastic liner or film fabricated from PTFE. In some embodiments, inner tubular surface 24 may be fabricated from any suitable plastic (e.g., silicone rubber, polyurethane, PET, thermoplastic polymers, and/or nylon). In embodiments, inner tubular surface 24 may have a durometer of about shore A65 to about shore A75. In some embodiments, inner tubular surface 24 may have a durometer of about shore A70.

In some embodiments, outer tubular surface 26 of elongated shaft 12 has a varying flexibility along its length to give each of proximal portion 12a, intermediate portion 12b, and distal portion 12c of elongated shaft 12 a distinct hardness, which facilitates navigation of elongated shaft 12 through the airways of the lung. In particular, in some embodiments, outer tubular surface 26 along proximal portion 12a of elongated shaft 12 has a durometer of about shore A70 to about shore A80 and, in some embodiments, a durometer of about shore A74. The outer tubular surface 26 along proximal portion 12a of elongated shaft 12 may be fabricated from a plastic material, such as, for example, polyphthalamide. In some embodiments, proximal portion 12a of outer tubular surface 26 may be fabricated from any suitable flexible material.

Outer tubular surface 26 along intermediate portion 12b of elongated shaft 12 has a durometer of about shore A35 to about shore A45 and, in some embodiments, a durometer of about shore A40. In embodiments where the portion of outer tubular surface 26 disposed along intermediate portion 12b of elongated shaft 12 is made of a more flexible material than the portion of outer tubular surface 26 disposed along proximal portion 12a of elongated shaft 12, intermediate portion 12b of elongated shaft 12 is more flexible than proximal portion 12a of elongated shaft 12. Outer tubular surface 26 along intermediate portion 12b of elongated shaft 12 may be fabricated from a plastic material, such as, for example, polyether block amide. In some embodiments, the portion of outer tubular surface 26 disposed along intermediate portion 12b of elongated shaft 12 may be fabricated from any suitable flexible material, including the same material as the portion of outer tubular surface 26 disposed along proximal portion 12a of elongated shaft 12 while still having a greater flexibility than the portion of the outer tubular surface 26 disposed along proximal portion 12a of elongated shaft 12.

The portion of outer tubular surface 26 disposed along distal portion 12c of elongated shaft 12 has a durometer of about shore A50 to about shore A60 and, in some embodiments, a durometer of about shore A55. As such, outer tubular surface 26 disposed along distal portion 12c may make distal portion 12c of elongated shaft 12 less flexible than intermediate portion 12b of elongated shaft 12, but more flexible than proximal portion 12a of elongated shaft 12. Less flexibility of distal portion 12c of elongated shaft 12 relative to intermediate portion 12b of elongated shaft 12 allows for distal portion 12c of elongated shaft 12 to be maintained in position relative to target tissue by being less susceptible to bending or flexing when abutting the target tissue.

With continued reference to FIGS. 3-7, elongated shaft 12 of catheter assembly 10 includes braiding 28 disposed between the inner and outer tubular surfaces 24, 26. Braiding extends longitudinally along proximal portion 12a of elongated shaft 12 and intermediate portion 12b of outer shaft, but terminates proximally of distal portion 12c of elongated shaft 12 such that distal portion 12c of elongated shaft 12 is devoid of braiding 28. In other embodiments, braiding 28 may extend longitudinally along proximal portion 12a, intermediate portion 12b, and distal portion 12c of elongated shaft 12. Braiding 28 includes a plurality of interwoven metallic braid filaments 28a, 28b, 28c arranged in a mesh-like configuration. Braiding 28 increases the structural integrity of elongated shaft 12 while allowing elongated shaft 12 to remain flexible. Each filament 28a-c of braiding 28 may be a flattened sheet or ribbon fabricated from a metal, for example, stainless steel. In some embodiments, braiding 28 may be fabricated from non-metallic filaments, for example, thermoplastic polymers.

With reference to FIGS. 1, 2 and 8-10D, needle shaft 40 of needle assembly 30 is configured to be moved within inner lumen 22 (FIGS. 2, 5, and 6) of elongated shaft 12 between a first position in which distal tip 42 of needle shaft 40 is disposed within elongated shaft 12 (FIGS. 1 and 2) and a second position in which distal tip 42 extends distally from distal portion 12c of elongated shaft 12 (FIGS. 8 and 9). Needle shaft 40 has a proximal portion 40a and a distal portion 40b and defines a longitudinally-extending lumen 44 therethrough. When needle shaft 40 is moved to the second position, biopsy system 1 (including the length of distal tip 42 and elongated shaft 12) may have an overall length of about 120 cm to about 150 cm and, in some embodiments, about 130 cm.

Hub 46 of needle assembly 30 may be fixedly or detachably coupled to proximal portion 40a of needle shaft 40. Hub 46 has a distal portion 46b configured for detachable coupling with handle portion 16 of hub 14. As such, when needle assembly 30 is moved to the second position (FIGS. 8 and 9), hub 46 of needle assembly 30 detachably couples to distal portion 16b of handle portion 16 of catheter assembly 10 to maintain needle shaft 40 in the second position relative to elongated shaft 12. Hub 46 of needle assembly 30 has a proximal portion 46a that may be in the form of a luer lock configured to form a fluid-tight connection with a syringe (not shown) and/or vacuum (not shown) for aspiration of a tissue sample.

Distal tip 42 of needle shaft 40 may have an overall length of between about 0.25 inches and about 0.5 inches. In some embodiments, distal tip 42 may have an overall length of about 0.325 inches. Distal tip 42 has a proximal portion 42a coupled to distal portion 40b of needle shaft 40 and a distal portion 42b in the form of a sharpened tip configured to pierce tissue. Proximal portion 42a of distal tip 42 is fixedly disposed within lumen 44 of needle shaft 40. In the illustrated embodiment, proximal portion 42a of distal tip 42 has an outer surface 48 with threading 50 defined therein, which assists in securing proximal portion 42a of distal tip 42 to distal portion 40b of needle shaft 40. The threading 50 has a truncated square pyramid cross-sectional shape having a width of between about 0.08 inches and about 0.012 inches and, in some embodiments, a width of about 0.010 inches. In some embodiments, threading 50 may have any suitable cross-sectional shape, such as, for example, triangular, square, rounded, or the like. In other embodiments, proximal portion 42a of distal tip 42 may be fastened to distal portion 40b of needle shaft 40 via any suitable fastening engagement, for example, adhesives, friction-fit, compression fit, interference fit, fasteners, or the like. It is contemplated that distal tip 42 may be integrally connected to or monolithically formed with distal portion 40b of needle shaft 40.

Distal portion 42b of distal tip 42 may be fabricated from metal (e.g., stainless steel) and defines a hollow interior 52 configured for receipt of tissue. Distal portion 42b of distal tip 42 has a lancet point configuration. It is contemplated that distal portion 42b of distal tip 42 may be any suitable needle tip type of any suitable geometry and any suitable gauge (e.g., 18 gauge) configured to pierce tissue.

With reference to FIGS. 11A-12C, biopsy system 1 may include a stylet 60 configured to be slidably received within lumen 44 of needle shaft 40. Stylet 60 includes a hub 62 and a shaft 64 extending distally from hub 62. Hub 62 includes an outer handle portion 66 and an inner tube 68 disposed within handle portion 66. Handle portion 66 of hub 62 may be a female luer connector configured to form a fluid tight seal with proximal portion 46a of hub 46 of needle shaft 40. In some embodiments, handle portion 66 of hub 62 may be any suitable connector (e.g., a threaded connector) for forming a detachable connection with hub 46 of needle shaft 40. Inner tube 68 of hub 62 extends distally from handle portion 66 of hub 62 and is configured to form a fluid-tight seal with lumen 44 (FIG. 2) of needle shaft 40 such that fluid may be passed through hub 62 and into lumen 44 when stylet 60 is coupled to needle shaft 40.

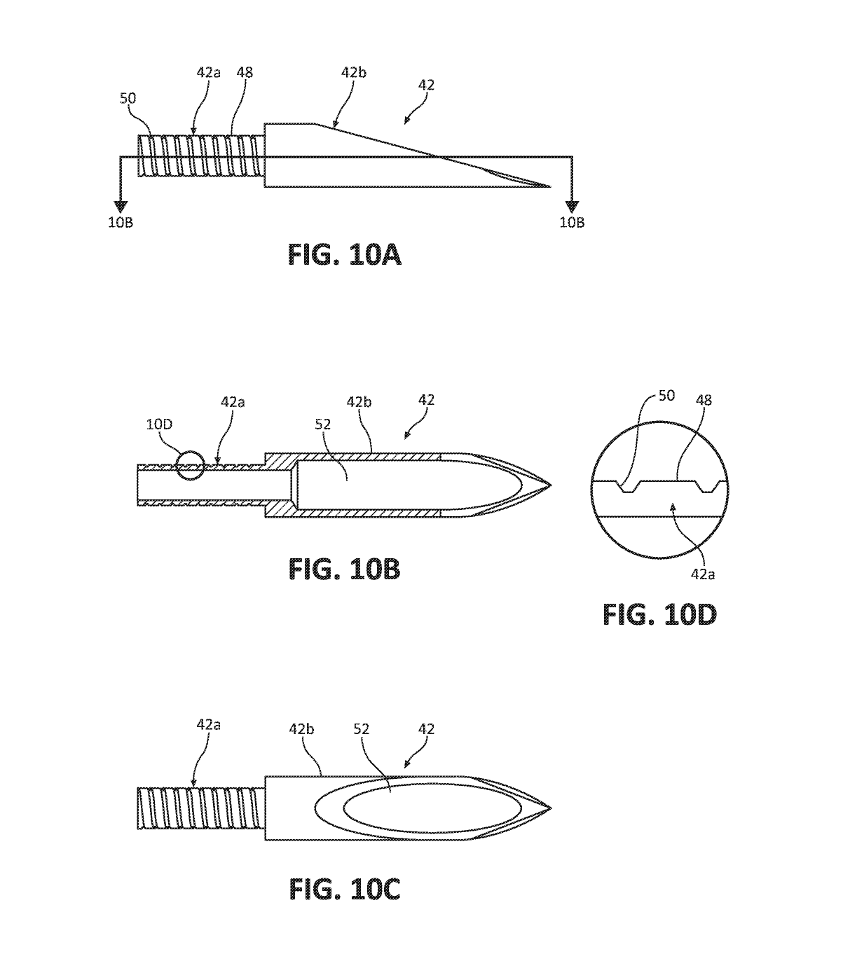

Shaft 64 of stylet 60 is disposed radially outward of inner tube 68 of hub 62 so as to not interfere with fluid passed between inner tube 68 of stylet 60 and lumen 44 of needle shaft 40. Stylet 60 terminates in a rounded distal tip 70 configured for receipt in hollow interior 52 (FIGS. 10B and 10C) of distal tip 42. Shaft 64 is configured so that upon locking hub 62 of stylet 60 to hub 46 of needle shaft 40, distal tip 70 of shaft 64 extends through hollow interior 52 of distal tip 42 to prevent distal tip 42 from cutting and receiving tissue until stylet 60 is withdrawn from hollow interior 52 of distal tip 42.

In operation of biopsy system 1, elongated shaft 12 is positioned through an access hole (e.g., a cannula or access port) to gain access into a lung of a patient. Elongated shaft 12 is navigated through the lung utilizing imaging guidance, for example, ultrasound, X-ray radiography, MRI, fluoroscopy, CT imaging, ENB, or the like, to position distal portion 12c of elongated shaft 12 adjacent lung tissue to be sampled. Methods used for navigating within the lung can be found in commonly assigned U.S. patent application Ser. No. 14/753,288, entitled “SYSTEM AND METHOD FOR NAVIGATING WITHIN THE LUNG,” filed on Jun. 29, 2015, the entire contents of which are incorporated herein by reference.

As elongated shaft 12 is guided through the various passageways of the lung, intermediate portion 12b of elongated shaft 12 bends to conform to the contours of the passageways of the lung. Needle shaft 40 of needle assembly 30, with shaft 64 of stylet 60 optionally disposed therein, is translated through lumen 22 of elongated shaft 12 to position distal tip 42 in the first position such that distal tip 42 does not extend distally from distal portion 12c, as shown in FIGS. 1 and 2. In some embodiments, needle shaft 40 and stylet 60 may be disposed within elongated shaft 12 as elongated shaft 12 is moved within the airways of the lung. Shaft 64 of stylet 60 may be used to increase the overall stiffness of elongated shaft 12. It is contemplated that stylet 60 may not be used at all during the procedure.

With distal portion 12c of elongated shaft 12 disposed adjacent the lung tissue to be sampled, a clinician may grasp handle portion 16 of catheter assembly 10 with one hand while pushing hub 46 of needle assembly 30 in a distal direction with the other hand to translate hub 46 of needle assembly 30 distally toward hub 14 of catheter assembly 10. As hub 46 of needle assembly 30 is translated distally relative to hub 14 of catheter assembly 10, distal tip 42 of needle shaft 40 moves through lumen 22 of elongated shaft 12. Continued distal movement of hub 46 of needle assembly 30 relative to hub 14 of catheter assembly 10 causes luer connector 46b of hub 46 to connect to handle portion 16 of hub 14 of catheter assembly 10, as shown in FIG. 8. Upon connecting hubs 14, 46 of respective catheter and needle assemblies 10, 30, distal tip 42 of needle shaft 40 is disposed in the second position such that distal tip 42 extends distally from distal portion 12c of elongated shaft 12, as shown in FIG. 9.

As distal tip 42 extends distally from distal portion 12c of elongated shaft 12, needle point 42b of distal tip 42 cuts through tissue and hollow interior 52 of distal tip 42 receives the cut tissue. Distal tip 42 may be rotated by rotating hub 46 of needle assembly 30 to facilitate removal of the tissue from the lung. A syringe, vacuum, or aspirator (not shown), attached to hub 46 of needle assembly 30, may be actuated to draw the sampled tissue into the distal tip 42. When utilizing stylet 60, as distal tip 42 extends distally from distal portion 12c of elongated shaft 12, blunt distal tip 70 of stylet 60 makes contact with the lung tissue prior to needle point 42b of distal tip 42 such that tissue is prevented from being received within distal tip 42 until stylet 60 is moved proximally out from hollow interior 52 of distal tip 42.

In some embodiments, after distal tip 42 captures the sample of lung tissue in hollow interior 52, needle shaft 40 and stylet 60 may be withdrawn from elongated shaft 12 and elongated shaft 12 may subsequently be removed from within the patient. In some embodiments, catheter assembly 10, needle assembly 30, and stylet 60 are simultaneously removed from within the patient. To remove the sampled tissue from distal tip 42, stylet 60 may be moved distally through hollow interior 52 of distal tip 42 to dislodge the sampled tissue from distal tip 42.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. A needle biopsy system, comprising:
an elongated shaft including:
a proximal portion;
an intermediate portion more flexible than the proximal portion; and
a distal portion less flexible than the intermediate portion; and
a needle shaft having a distal tip configured to cut and receive a sample of tissue, the needle shaft longitudinally movable through the elongated shaft between a first position in which the distal tip of the needle shaft is disposed within the elongated shaft and a second position in which the distal tip of the needle shaft extends distally from the distal portion of the elongated shaft to cut and receive a sample of tissue.

2. The needle biopsy system according to claim 1, wherein the elongated shaft has an outer tubular surface, an inner tubular surface, and a braiding extending longitudinally between the outer tubular surface and the inner tubular surface.

3. The needle biopsy system according to claim 2, wherein the braiding extends along the proximal and distal portions of the elongated shaft and terminates proximally of the distal portion of the elongated shaft.

4. The needle biopsy system according to claim 2, wherein the braiding includes a plurality of interwoven metallic braid filaments.

5. The needle biopsy system according to claim 2, wherein the inner tubular surface has a constant durometer along its length.

6. The needle biopsy system according to claim 2, wherein the outer tubular surface has a higher durometer along the proximal portion of the elongated shaft than at the intermediate portion and the distal portion of the elongated shaft.

7. The needle biopsy system according to claim 2, wherein the proximal portion of the elongated shaft is less flexible than the intermediate portion and the distal portion of the elongated shaft.

8. The needle biopsy system according to claim 1, wherein the distal portion of the elongated shaft is tapered in a distal direction.

9. The needle biopsy system according to claim 1, further comprising:
a first hub coupled to the proximal portion of the elongated shaft and configured to receive the needle shaft therethrough; and
a second hub coupled to a proximal portion of the needle shaft, the needle shaft configured to move through and relative to the first hub in response to movement of the second hub.

10. The needle biopsy system according to claim 9, wherein the first hub has a proximal portion configured to detachably engage a distal portion of the second hub.

11. The needle biopsy system according to claim 9, wherein the second hub is configured for a fluid tight connection with a syringe.

12. The needle biopsy system according to claim 1, further comprising a stylet configured to move longitudinally within a lumen defined through the needle shaft.

13. The needle biopsy system according to claim 12, wherein the stylet includes a blunt distal tip configured to be disposed within the distal tip of the needle shaft.

14. The needle biopsy system according to claim 12, wherein the stylet is less flexible than the intermediate portion of the elongated shaft.

15. A method of performing a needle biopsy of lung tissue, comprising:
flexing at least a portion of an elongated shaft to facilitate moving the elongated shaft through an airway of a lung;
positioning a blunt distal portion of the elongated shaft adjacent tissue in the lung, an intermediate portion of the elongated shaft being more flexible than a proximal portion of the elongated shaft and the blunt distal portion being less flexible than the intermediate portion and more flexible than the proximal portion; and
moving a needle shaft longitudinally within the elongated shaft to extend a distal tip of the needle shaft from the distal portion of the elongated shaft to cut and receive a sample of tissue.

16. The method according to claim 15, further comprising moving a blunt distal tip of a stylet in a proximal direction to a position proximal of the distal tip of the needle shaft.

17. The method according to claim 15, further comprising moving a stylet longitudinally within a lumen defined through the needle shaft to position a distal tip of the stylet within the distal portion of the needle shaft.
